(19) United States
(12) Patent Application Publication
(10) Pub. No.: US 2006/0141415 A1
(43) Pub. Date: Jun. 29, 2006

(54) Intra-oral mirrors

(76) Inventors: Ryan Johnson, Denver, CO (US); Breanne Sanger, Denver, CO (US)

Correspondence Address: TRENNER LAW FIRM, LLC, 12081 WEST ALAMEDA PARKWAY #163, LAKEWOOD, CO, 80228, US

(21) Appl. No.: 11/345,583

(22) Filed: Feb. 1, 2006

Related U.S. Application Data
(60) Provisional application No. 60/738,285, filed on Nov. 18, 2005.

(51) Int. Cl. A61B 1/24; A61C 5/00

(52) U.S. Cl. 433/30; 433/140

(57) ABSTRACT

Mirrors for viewing and/or photographing occlusal, buccal, and lingual views of a patient's teeth are described. In an exemplary embodiment, a mirror comprises a first lobe providing an imaging surface during use. The first lobe has a reflective surface for reflecting a photo-quality image of the patient's teeth in the direction of a camera approach during use. A second lobe provides a handle. The first and second lobes are connected to one another and form an angle between the handle and the imaging surface that automatically positions a user's hand on the handle away from the camera approach during use.

### PRIORITY CLAIM

This application claims priority to co-owned U.S. Provisional Patent Application No. 60/738,285 for “INTRA-ORAL MIRROR” of Johnson, et al. (Attorney Docket No. NOVUS.001.PRV), filed Nov. 18, 2005, hereby incorporated by reference in its entirety as though fully set forth herein.

### TECHNICAL FIELD

The described subject matter relates to oral or dental products in general, and more particularly to intra-oral mirrors.

### BACKGROUND

One of a dentist's toughest jobs is explaining to a patient the need for dental treatment and having the patient understand that such treatment is necessary. Although the dentist may explain one or more dental problems to the patient and potential consequences of failing to treat these problems, patients are often reluctant to rely on someone else's word.

Although the dentist may show the patient x-rays of the patient's mouth, x-rays are often difficult for the untrained eye to understand. Dental mirrors are also commercially available. However, dental mirrors are typically configured such that the dentist can more readily view teeth toward the back of the patient's mouth and/or the inside surfaces of the patient's teeth. The images on these mirrors often cannot be readily viewed by the patient. If the user tries to hold larger, flat mirrors in the patient's mouth, his or her fingers tend to obstruct the image. Cheek retractors may also be needed to effectively use larger, flat mirrors.

Dentists may also use a camera to photograph the patient's teeth and show the photograph to the patient. However, most conventionally available cameras are too large to fit into the patient's mouth, limiting the area that can be photographed to a view from the outside of the patient's mouth, similar to what tile patient could see by standing in front of a mirror.

### SUMMARY

An exemplary intra-oral mirror may comprise a first lobe providing an imaging surface during use, the first lobe having a reflective surface for reflecting a photo-quality image of the patient's teeth in the direction of a camera approach during use. A second lobe provides a handle. The first and second lobes are connected to one another and form an angle between the handle and the imaging surface that automatically positions a user's hand on the handle away from the camera approach during use.

An exemplary method may comprise: providing a mirror for partial insertion into a patient's mouth, the mirror having opposing lobes oriented at an angle relative to one another such that a user's hand is positioned away from a camera approach during use even if the user is holding the mirror in the patient's mouth, and reflecting an image of a patient's teeth from one of the opposing lobes toward the camera approach when the other opposing lobe is positioned in the patient's mouth adjacent the patient's teeth, the image of the patient's teeth suitable for photographing.

### BRIEF DESCRIPTION OF THE DRAWINGS

FIG. 1 is a diagrammatic plan view illustrating a patient's top and bottom set of teeth.

FIG. 2 is a perspective view of an exemplary occlusal mirror.

FIG. 2a is a top plan view of the occlusal mirror shown in FIG. 2. FIG. 2b is a side elevation view of the exemplary occlusal mirror shown in FIG. 2. FIG. 2c is a side elevation view of an exemplary alternative embodiment of the occlusal mirror.

FIG. 2d is a top plan view of the occlusal mirror shown in FIG. 2 showing an exemplary offset. FIG. 2e is a top plan view of an alternative occlusal mirror to the mirror shown in FIG. 1 showing another exemplary offset.

FIGS. 3a and 3b are diagrammatic side views of a patient's mouth illustrating implementation of an exemplary occlusal mirror as it may be used for photographing a patient's (a) top teeth and (b) bottom teeth.

FIGS. 4a and 4b are perspective views of a patient's mouth illustrating an exemplary occlusal mirror as it may be used for photographing (a) top teeth and (b) bottom teeth.

FIG. 5 is a perspective view of an exemplary buccal mirror.

FIG. 5a is a top plan view of the buccal mirror shown in FIG. 5. FIG. 5b is a side elevation view of the exemplary buccal mirror shown in FIG. 5. FIG. 5c is a side elevation view of an exemplary alternative embodiment of the buccal mirror.

FIGS. 6a and 6b are diagrammatic plan views of a patient's mouth illustrating implementation of an exemplary buccal mirror as it may be used for photographing a patient's (a) right-side teeth and (b) left-side teeth.

FIGS. 7a and 7b are perspective views of a patient's mouth illustrating an exemplary buccal mirror as it may be used for photographing the patient's (a) right-side teeth and (b) left-side teeth.

FIG. 8 is a perspective view of an exemplary lingual mirror.

FIG. 8a is a top plan view of the exemplary lingual mirror shown in FIG. 8. FIG. 8b is a side elevation view of the exemplary lingual mirror shown in FIG. 8. FIG. 8c is a side elevation view of an exemplary alternative embodiment of the lingual mirror.

FIGS. 9a and 9b are diagrammatic plan views of a patient's mouth illustrating implementation of an exemplary lingual mirror as it may be used for photographing a patient's teeth in (a) the top-right quadrant, and (b) the bottom-right quadrant.

FIGS. 10a and 10b are perspective views of a patient's mouth illustrating all exemplary lingual mirror as it may be used for photographing the inner side-surface of a patient's teeth in (a) the top-right quadrant, and (b) the bottom-right quadrant.

### DETAILED DESCRIPTION

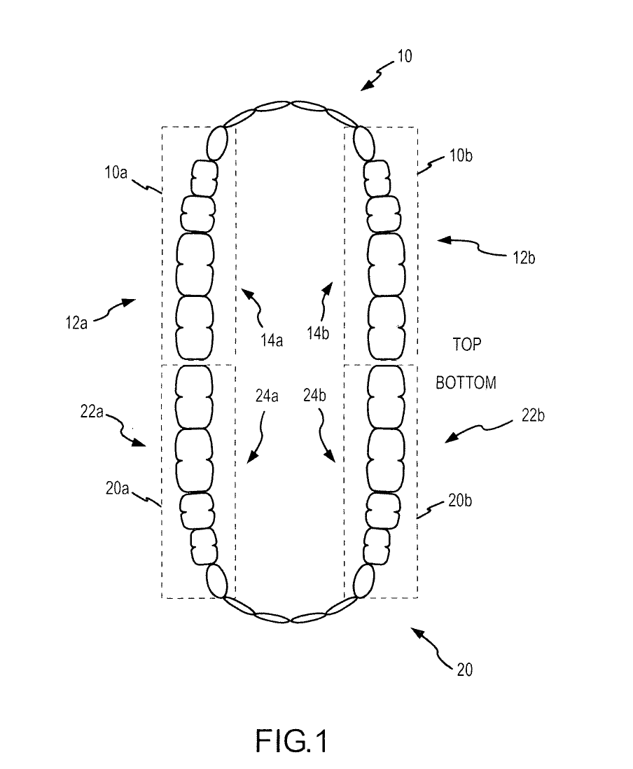

FIG. 1 is a diagrammatic top plan view of a patient's maxillary (upper) set of teeth 10 and mandibular (bottom) set of teeth 20. The intra-oral mirrors shown and described herein enable a user to readily see and/or photograph occlusal, buccal, and lingual views of a patient's teeth, as will be explained in more detail below with reference back to FIG. 1.

Exemplary mirrors are easy to use, comfortable for the patient, and cost-effective. The configurations allow the mirrors to be comfortably positioned in the patient's mouth and grasped during image capture without the user's hand interfering with the camera approach. The configurations also allow the same mirrors to be used with different size mouths (e.g., adult/child or male/female).

In exemplary embodiments, the mirrors may be used with conventionally available cameras to photograph a patient's teeth and/or inside of the patient's mouth, enabling dentists to better explain the need for dental work (e.g., fillings, crowns, implants, extractions and other dental work). For example, a dentist can show a patient a picture of the patient's teeth and/or inside of the patient's mouth so that the patient can see for him or herself the problems, such as, stress fractures, decay, and advanced periodontal problems. By seeing and understanding the problem firsthand, the patient is more likely to understand and accept the recommended treatment. The dentist can also use the photographs to better document the need for temporary and permanent restorations in a patient's chart, show another dentist or specialist for a second opinion, and/or send the photographs to insurance companies when their approval is required in order to cover the costs of treatment.

These and other advantages will be readily apparent to one having ordinary skill in the art after becoming familiar with the teachings herein.

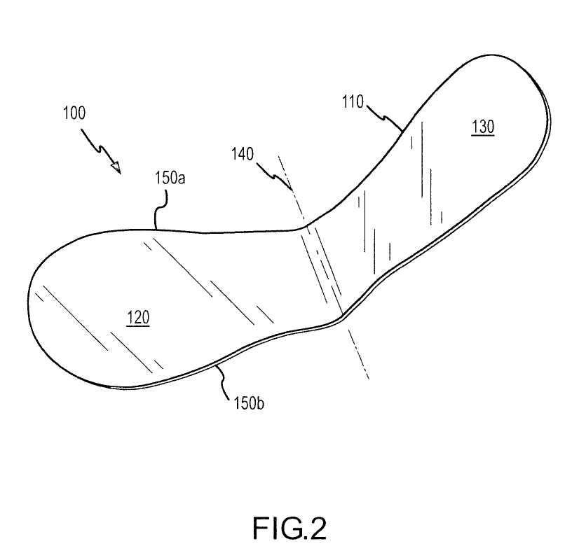

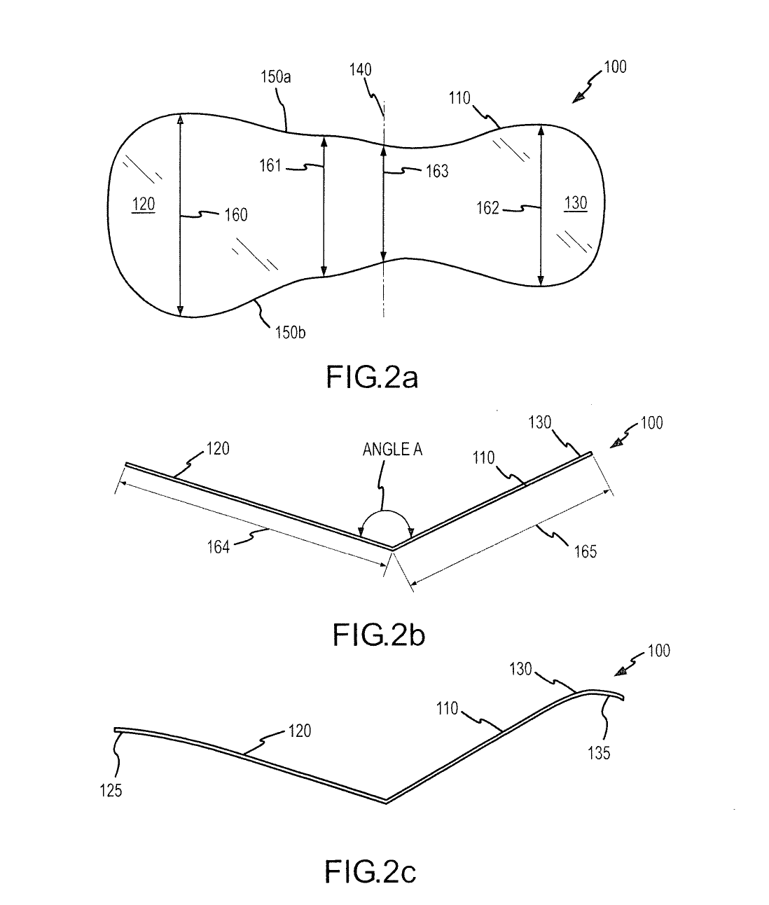

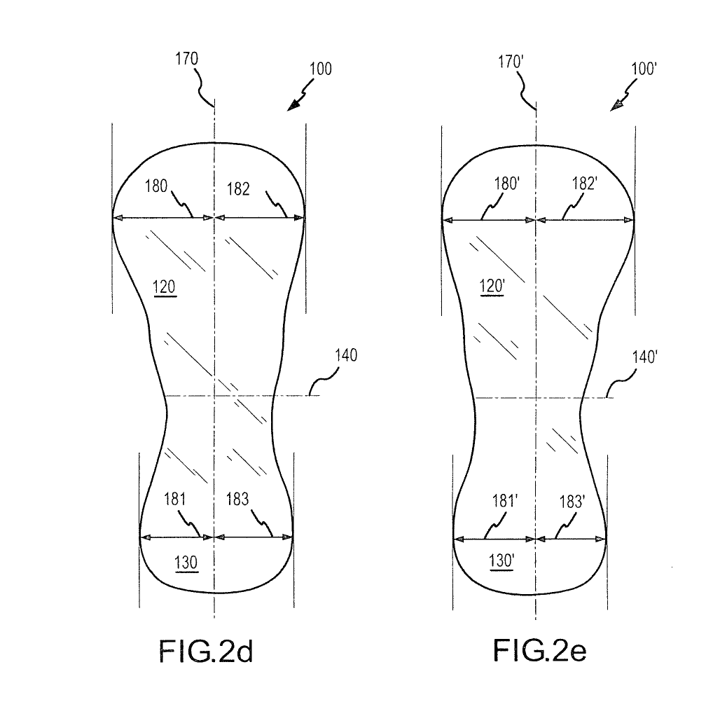

FIG. 2 is a perspective view of all exemplary occlusal mirror 100. FIG. 2a is a top plan view of the occlusal mirror 100 shown in FIG. 2. FIG. 2b is a side elevation view of the exemplary occlusal mirror 100 shown in FIG. 2.

Occlusal mirror 100 may include one or more reflective surfaces. In all exemplary embodiment, the entire body 110 of occlusal mirror 100 is made of (or otherwise provided with) a reflective surface. Optionally, all sides of the body 110 are reflective so that the occlusal mirror 100 is reversible. For example, the occlusal mirror 100 may be manufactured of a stainless steel material and polished for a reflective finish. Stainless steel construction helps reduce or altogether prevent scratching and discoloration of the reflective surfaces (e.g., during sterilization in all autoclave). In addition, stainless steel construction enables sterilization by heat (e.g., autoclave) or chemical wash.

It is noted, however, that the occlusal mirror or 100 is not limited to manufacture from any type of material. For example, occlusal mirror 100 may be manufactured of plastic and coated with a reflective surface (e.g., a chrome or rhodium finish).

It is also noted that the entire surface of occlusal mirror 100 need not have a reflective surface. For example, one or more reflective surfaces may be attached to the body 110.

The body 110 of occlusal mirror 100 may be configured as first and second lobes 120, 130. The body 110 may be formed with a bend along line 140 in FIG. 2 (which can also be seen in FIGS. 2a-2e). Optionally, the body 110 may also be provided with indentations or notches (e.g., referred to by arrows 150a, 150b in FIGS. 2 and 2a).

In an exemplary embodiment, the first and second lobes 120, 130 form a substantially “figure-eight” shaped body 110, e.g., as can be seen in FIGS. 2 and 2a. In other embodiments, however, the first and/or second lobes may be other shapes.

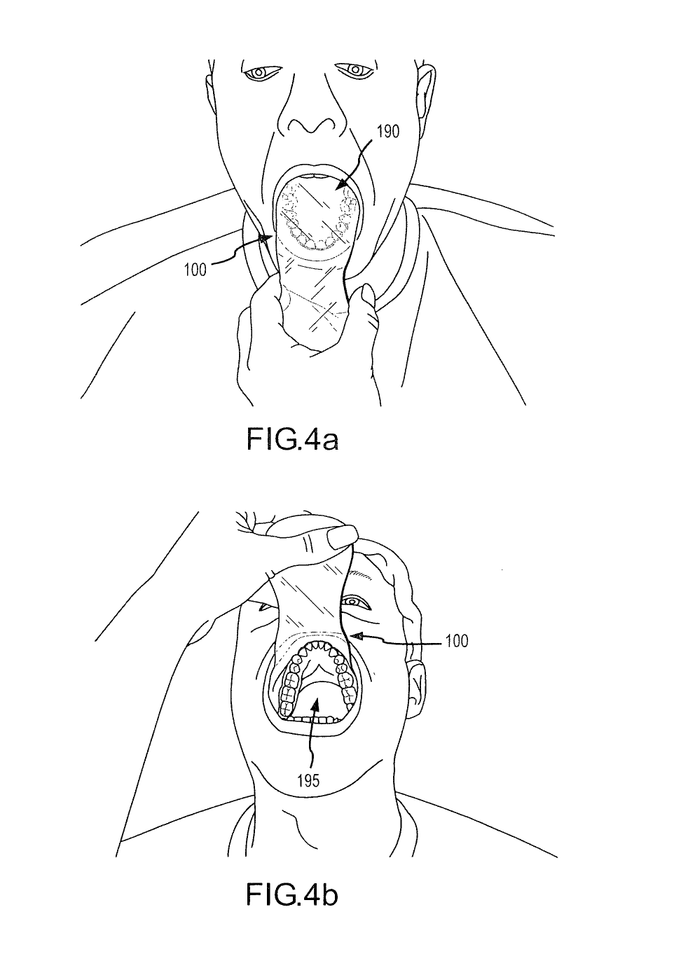

The first lobe 120 may be used as a handle and the second lobe 130 may provide an imaging surface. That is, a user may grasp the first lobe 120 and insert the second lobe 130 of the occlusal mirror 100 in a patient's mouth. The patient's teeth (and/or surrounding areas of the mouth) are reflected by the imaging surface and can be viewed and/or photographed, as explained in more detail below with reference to FIGS. 4a and 4b. In another exemplary embodiment, first lobe 120 may provide the imaging surface and second lobe 130 may be used as the handle. Accordingly, occlusal mirror 100 may be used interchangeably.

It is noted that the first and second lobes 120, 130 may be rounded and/or polished to enhance patient comfort during use (e.g., reducing gag reflex). The edges of the body 110 may also be polished to reduce cuts or other discomfort to the patient.

Although the first and second lobes 120, 130 may be the same size, or about the same size, in an exemplary embodiment the first lobe 120 is larger in size than the second lobe 130. According to such an embodiment, the larger first lobe 120 may provide the imaging surface when the occlusal mirror 100 is being used for patient's having larger mouths (e.g., for adult use), and the second lobe 130 may be used as the handle. In addition, the smaller second lobe 130 may provide the imaging surface when the occlusal mirror 100 is being used for patient's having smaller mouths (e.g., for pediatric use), and the first lobe 120 may be used as the handle.

In this manner, the same mirror may be used for patients having different size mouths, reducing the number of mirrors the manufacturer has to stock (and the dentist office has to purchase). In addition, if the user tries one size mirror and determines that the mirror is too large (or small) for the patient, the user can turn it around and try the other size (larger or smaller) mirror. After use, only one mirror has to be sterilized because only one mirror was used.

For purposes of illustration, an exemplary interchangeable adult male and pediatric occlusal mirror may have the following dimensions. With reference to FIG. 2a, the width of first lobe 120 at its widest part (referred to by dimension arrows 160) may be about two and three-quarter inches and the width of the first lobe 120 at notches indicated by arrows 150a, 150b (referred to by dimension arrows 161) may be about two inches. The width of second lobe 130 at its widest part (referred to by dimension arrows 162) may be about two inches. The width at the bend indicated by line 140 in FIGS. 2 and 2a (referred to by dimension arrows 163) may be about one and seven-eighths inches. With reference now to FIG. 2b, the length of first lobe 120 (referred to by dimension arrows 164) may be about four inches and the length of second lobe 130 (referred to by dimension arrows 165) may be about three inches.

Also for purposes of illustration, an exemplary interchangeable adult female and pediatric occlusal mirror may have the following dimensions. Again with reference to FIG. 2a, the width of first lobe 120 at its widest part (referred to by dimension arrows 160) may be about two and one-quarter inches and the width of the first lobe 120 at notches indicated by arrows 150a, 150b (referred to by dimension arrows 161) may be about one and five-eighths inches. The width of second lobe 130 at its widest part (referred to by dimension arrows 162) may be about two inches. The width at the bend indicated by line 140 in FIGS. 2 and 2a (referred to by dimension arrows 163) may be about one and one-quarter inches. With reference now to FIG. 2b, the length of first lobe 120 (indicated by dimension arrows 164) may be about three and three-quarter inches and the length of second lobe 130 (indicated by dimension arrows 165) may be about two and one-half inches.

It is noted that the occlusal mirror 100 is not limited to any particular dimensions. For example, other dimensions may be selected, such as for use as an interchangeable adult and teenager mirror. Of course, occlusal mirrors 100 are not limited to “generic” sizes (e.g., adult, teenager, male, female, pediatric). For example, the mirrors may also be custom sized. Still, in other embodiments the occlusal mirrors 100 do not need to be interchangeable between different sizes.

Although the occlusal mirror 100 is not limited to any particular size or shape, the size of lobes 120, 130 may be selected to comfortably fit in the mouth of patients, while providing a photo-quality image of the rear-most (e.g., third) molars on each side of the patient's mouth.

As briefly mentioned above, the body 110 may also be provided with indentations or notches (e.g., referred to by arrows 150a, 150b in FIG. 2a). Although the notches are only shown in FIG. 2a as being formed in the larger lobe 120, it is noted that in other embodiments, the notches may be formed in the smaller lobe 130 instead of, or in addition to, being formed in the larger lobe 120.

The notches enable the user to better grasp the lobe 120 when it is being used as a handle. The notches also allow the lobe 120 to be more readily inserted into the patient's mouth when it is being used as the imaging surface. In addition, the patient is able to partially close his or her mouth around the notches for a more comfortable fit when the lobe 120 having notches is inserted in the patient's mouth.

Also as briefly mentioned above, the body 110 may be formed with a bend along line 140 in FIG. 2 (also shown in FIGS. 2a-2e). The angle of this bend (referred to as angle A in FIG. 2b) may be selected such that when the user is holding one of the lobes 120, 130, the user's hand is automatically positioned away from the imaging surface and does not interfere with the camera approach, as illustrated in FIGS. 4a and 4b. That is, the bend enables the user to grasp the handle and hold the occlusal mirror 100 so that the imaging surface is in position for the desired image of the patient's teeth and surrounding portions of the mouth. The user does not have to move his or her hand to photograph the reflection of the patient's teeth (and surrounding portions of the mouth) on the imaging surface.

It is noted that although experimental use has shown that an angle A (shown in FIG. 2b) of about 135 degrees effectively accomplishes the advantages discussed above, the occlusal mirror 100 is not limited to being manufactured and used with this angle. The mirror may be manufactured for use with other angles (e.g., an angle A of 150 degrees) which will depend at least to some extent on design considerations, such as, e.g., the size of the mirror, the size of the user's hand, the size of the patient's mouth, and the desired image, to name only a few examples.

Although the first and second lobes 120, 130 of occlusal mirror 100 may be substantially planar, as can be seen in FIG. 2b, the occlusal mirror 100 is not limited such a configuration. For example, FIG. 2c is a side elevation view of an alternative embodiment of the occlusal mirror, wherein the lobes 120, 130 are curved at the outermost edges indicated at 125 and 135, respectively. Such an embodiment may further enhance patient comfort during use. Still other embodiments, not shown, are also contemplated.

Although in exemplary embodiments, the body 110 of occlusal mirror 100 may be symmetrical about a longitudinal centerline 170 shown in FIG. 2d (and 170′ shown in FIG. 2e), the occlusal mirror 100 is not limited to such a configuration. For example, FIGS. 2d and 2e are top plan views of two different occlusal mirrors showing the lobes being offset from the longitudinal centerline 170.

The embodiment shown in FIG. 2d corresponds to occlusal mirror 100 shown in FIG. 2. In this embodiment, the lobes 120, 130 are wider on one side of longitudinal centerline 170. The offset can be seen by comparing the longer distance from the longitudinal centerline to the edge on one side of the lobes 120, 130 (indicated by arrows 180 and 181) with the shorter distance from the longitudinal centerline to the edge on the other side of the lobes 120, 130 (indicated by arrows 182 and 183). This embodiment may be implemented, e.g., by right-handed users, because it may give the right-handed user a better grip.

The occlusal mirror 100′ shown in FIG. 2e is a “mirror image” of the occlusal mirror 100 shown in FIG. 2d. That is, the lobes 120′, 130′ are wider on the opposite side of longitudinal centerline 170′, as can be seen by comparing the shorter distances indicated by arrows 180′ and 181′ to the longer distances indicated by arrows 182′, 183′. This embodiment may be implemented, e.g., by left-handed users, because it may give the left-handed user a better grip.

In an exemplary embodiment, the offset also enables the user to implement the occlusal mirror 100 or 100′ as a retractor. That is, the larger portion of the lobe 120 or 130 (indicated by distance 180, 181 and 182′, 183′) may be used to “hook” the lobe 120 or 130 in a patient's mouth, retract the patient's cheek, and then slide the remainder of the lobe into the patients mouth. This helps make entry and exit of the occlusal mirror 100 or 100′ into and out of the patient's mouth more comfortable as opposed to forcing the lobe 120 or 130 straight into the patient's mouth. It also reduces or altogether eliminates the need for cheek retractors.

Exemplary embodiments of the occlusal mirror shown and described above may be implemented to obtain occlusal views of a patient's teeth (and surrounding gum and other parts of the mouth). One exemplary occlusal view reflected by the imaging surface of occlusal mirror 100 includes a full arch view of the top surfaces of the patient's maxillary teeth 10, as seen in the top portion of FIG. 1. Another exemplary occlusal view reflected by the imaging surface of occlusal mirror 100 includes a full arch view of the top surfaces of the patients mandibular teeth 20, as seen in the bottom portion of FIG. 1.

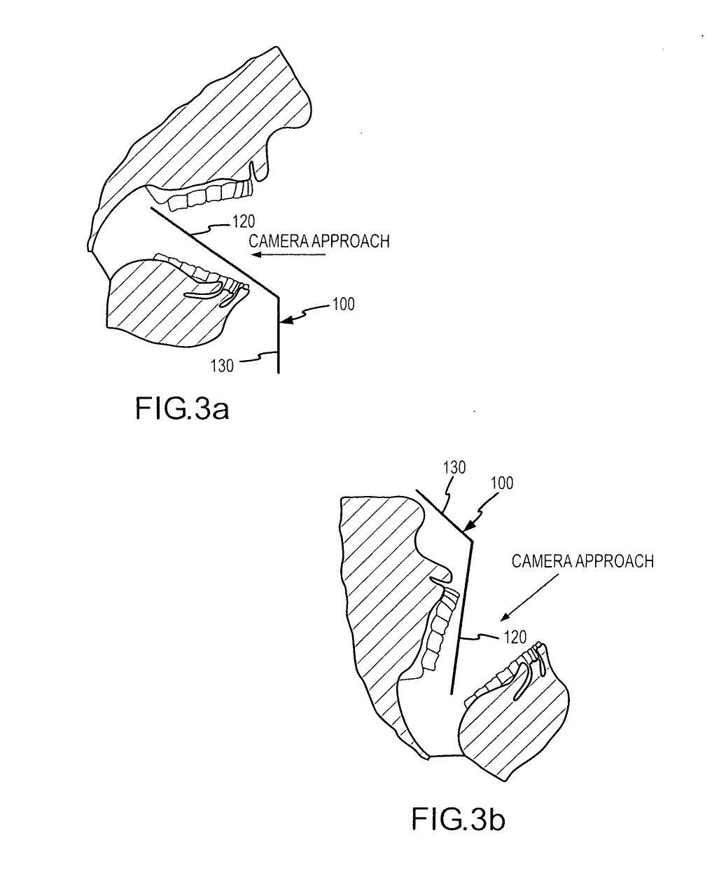

Occlusal views, such as those described above, may be viewed and/or photographed using the occlusal mirror 100 as illustrated in FIGS. 3a-3b and FIGS. 4a-4b. FIGS. 3a and 3b are diagrammatic side views of a person's mouth illustrating implementation of an exemplary occlusal mirror as it may be used for photographing an occlusal view of a patient's (a) top teeth and (b) bottom teeth. FIGS. 4a and 4b are perspective views of a person's mouth corresponding to FIGS. 3a and 3b, illustrating implementation of an exemplary occlusal mirror as it may be used for photographing an occlusal view of a patient's (a) top teeth and (b) bottom teeth.

The user (e.g., dentist, dental technician or even the patient) may hold the occlusal mirror 100 by one of the lobes 120 or 130, and insert the other lobe (i.e., the imaging surface) into the patient's mouth. The imaging surface reflects an occlusal view of the patient's teeth (and surrounding gums and mouth). For a maxillary image (i.e., top teeth), the user may rest the mirror on the lower lip or front teeth, as shown in FIG. 4a. For a mandibular image (i.e., bottom teeth), the user may press the mirror against the upper lip or front teeth, as shown in FIG. 4b.

The user (or another user such as an assistant) may position a camera as illustrated by the camera approach in FIGS. 3a and 3b to photograph the reflected image 190 and 195 in FIGS. 4a and 4b, respectively. As mentioned above, the bend about line 140 (see, e.g., FIG. 2) automatically positions the user's fingers such that they do not interfere with the reflected image. The user does not have to maneuver his or her fingers out of the way of the camera approach.

It is noted that the occlusal mirror 100 may be used to obtain full arch occlusal views, or partial occlusal views, as will be readily appreciated by the user by positioning the occlusal mirror in a patient's mouth and seeing the different views.

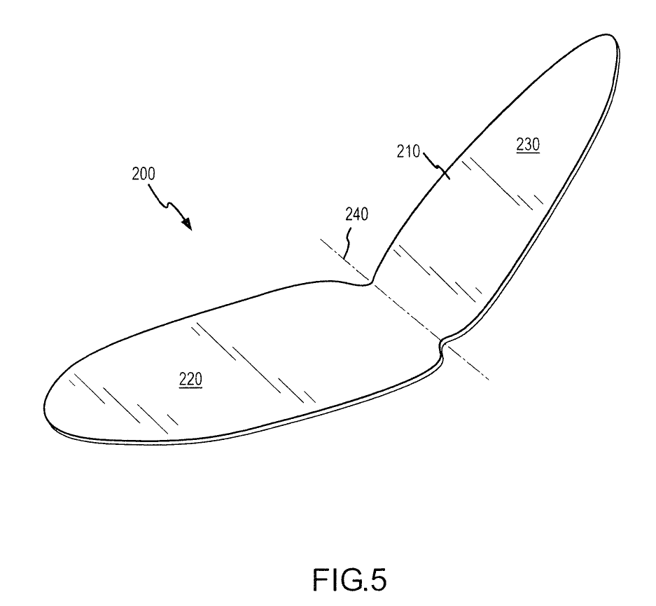

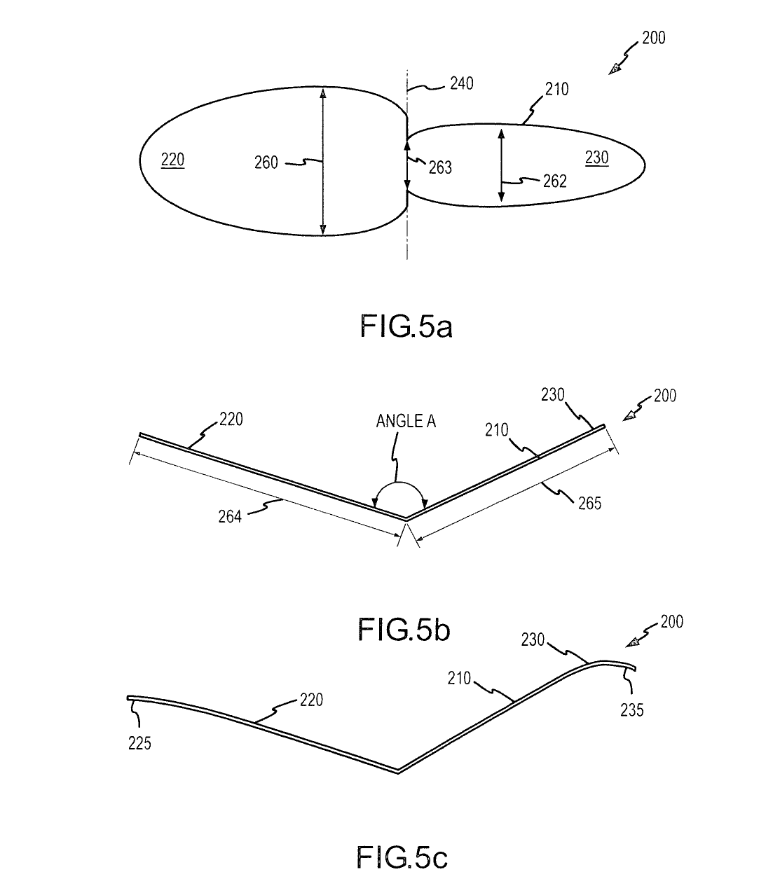

FIG. 5 is a perspective view of an exemplary buccal mirror 200. FIG. 5a is a top plan view of the buccal mirror 200 shown in FIG. 5. FIG. 5b is a side elevation view of the exemplary buccal mirror 200 shown in FIG. 5.

It is noted that many features described above for the occlusal mirror 100 are also applicable to the buccal mirror 200, and therefore the descriptions are not repeated herein for the buccal mirror 200. Instead, the following discussion explains features of the buccal mirror 200 while incorporating the above discussion of the occlusal mirror 100 by reference. It is also noted that 200-series references are used to refer to like elements designated by the 100-series references above, and may not be fully described again with regard to the buccal mirror 200.

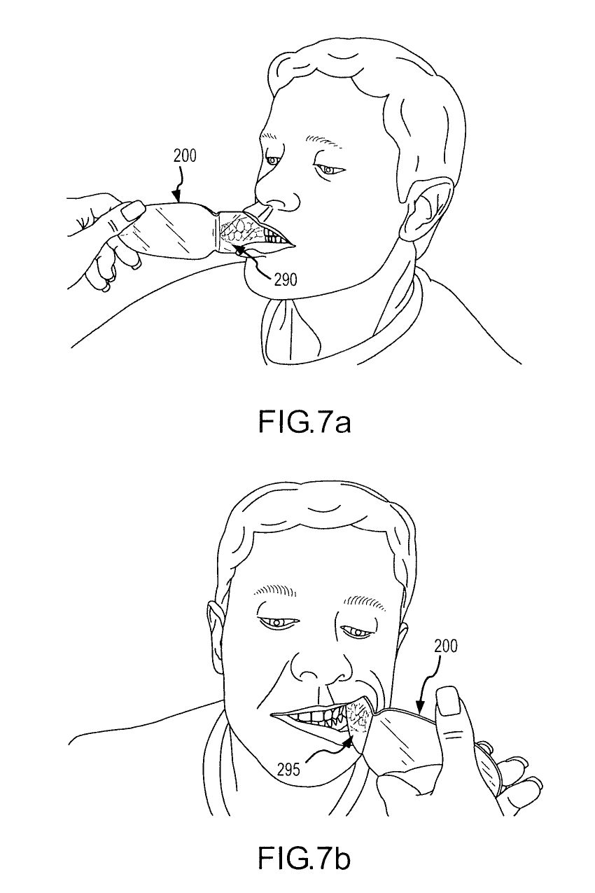

As described above for the occlusal mirror 100, buccal mirror 200 may comprise a body 210 with first and second lobes 220, 230. In an exemplary embodiment, the first and second lobes of the buccal mirror 200 are substantially oval or “tear-drop” shape, (e.g., as can be seen in FIGS. 5 and 5a) to fit along the side of the patient's mouth (see, e.g., FIGS. 7a and 7b).

For purposes of illustration, an exemplary interchangeable adult male and pediatric occlusal mirror may have the following dimensions. With reference to FIG. 5a, the width of first lobe 220 at its widest part (referred to by dimension arrows 260) is about one and seven-eighths inches. The width of second lobe 230 at its widest part (referred to by dimension arrows 262) may be about one and one-quarter inches. The width at the bend indicated by line 240 in FIGS. 5 and 5a (referred to by dimension arrows 263) may be about one and one-eighths inches. With reference now to FIG. 5b, the length of first lobe 220 (indicated by dimension arrows 264) may be about three and seven-sixteenth inches and the length of second lobe 230 (indicated by dimension arrows 265) may be about three inches. Angle A is about 135 degrees.

As with the occlusal mirror 100, it is noted that other embodiments of the buccal mirror 200 are not limited to any particular shape, size, or angle A, and do not need to be interchangeable between different sizes.

Although not shown, in other embodiments the buccal mirror 200 may also include indentations or notches similar to those described above for the occlusal mirror 100. Likewise, the buccal mirror 200 may be symmetrical about a longitudinal centerline, as shown in the figures. In alternative embodiments, however, the buccal mirror 200 may includes features which are offset from the longitudinal centerline similarly to that described above for the occlusal mirror 100, e.g., to enhance use as a retractor and provide a better grip for right-handed or left-handed users.

In addition, the first and second lobes 220, 230 of buccal mirror 200 may be substantially planar, as can be seen in FIG. 5b, or curved at the outermost edges indicated at 225 and 235, respectively, as shown in an alternative embodiment of the buccal mirror in FIG. 5c.

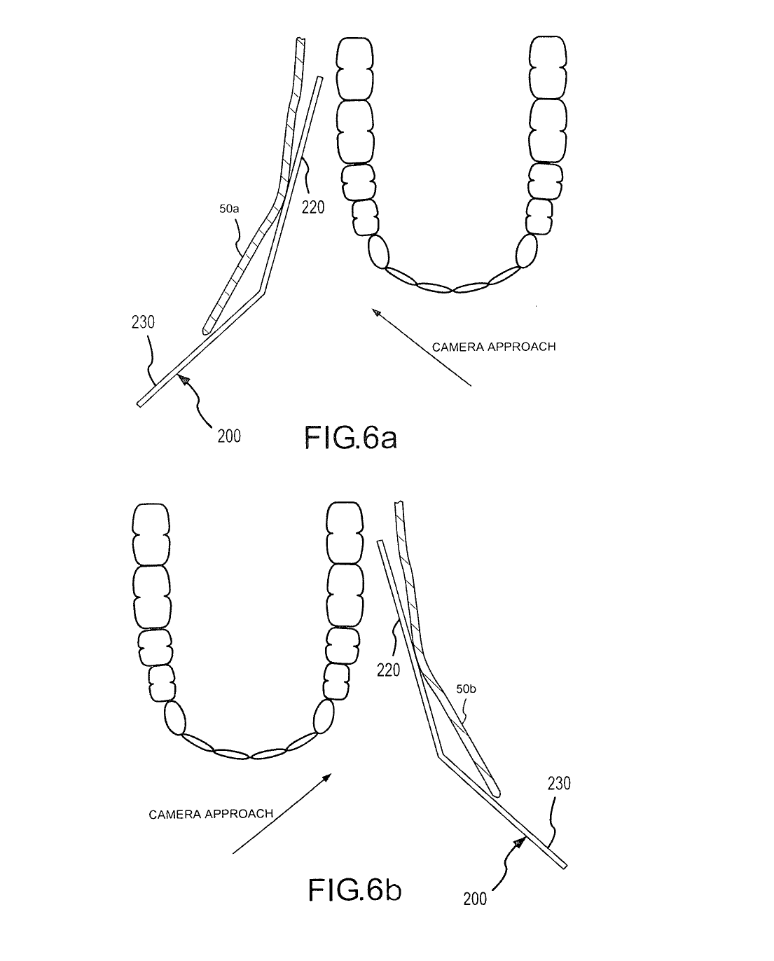

FIGS. 6a and 6b are diagrammatic plan views of a patient's mouth illustrating implementation of an exemplary buccal mirror as it may be used for photographing a patient's (a) right-side teeth and (b) left-side teeth. FIGS. 7a and 7b are perspective views of a person's mouth, illustrating implementation of an exemplary buccal mirror 200 as it may be used for photographing a buccal view of a patient's (a) right-side teeth and (b) left-side teeth.

Buccal views are views of the left-outside and right-outside surfaces of a patient's teeth (and surrounding gum and other parts of the mouth), e.g., as indicated in FIG. 1 by arrows 12a and 22a (right-outside) and arrows 12b and 22b (left-outside).

In use, the user (e.g., dentist, dental technician or even the patient) may hold the buccal mirror 200 by one of the lobes 220 or 230, and insert the other lobe (i.e., the imaging surface) into the patient's mouth, and pull back to retract the patient's cheek (shown diagrammatically by reference 50a in FIG. 6a and reference 50b in FIG. 6b). The imaging surface reflects a buccal view of the patient's teeth (and surrounding gums and mouth). The bend automatically positions the user's fingers such that they do not interfere with the image being reflected by the imaging surface. The user does not have to maneuver his or her hand out of the way of the camera approach.

The user (or another user such as an assistant) may position a camera adjacent the buccal mirror 200 to photograph the image being reflected by the mirror 200, as indicated by the camera approach arrows in FIGS. 6a and 6b. Both the left and right side images of a patient's mouth can be readily photographed can be seen in FIGS. 7a and 7b.

It is noted that the buccal mirror 200 may be used to obtain separate buccal views of each quadrant (e.g., quadrants 10a, 10b, 20a, and 20b in FIG. 1) by having the patient open his or her mouth and holding the buccal mirror 200 adjacent each quadrant for separate images. Alternatively, the buccal mirror 200 may be used to obtain combined top and bottom buccal views of each side of the patient's mouth by having the patient close his or her mouth, as shown in FIGS. 7a and 7b.

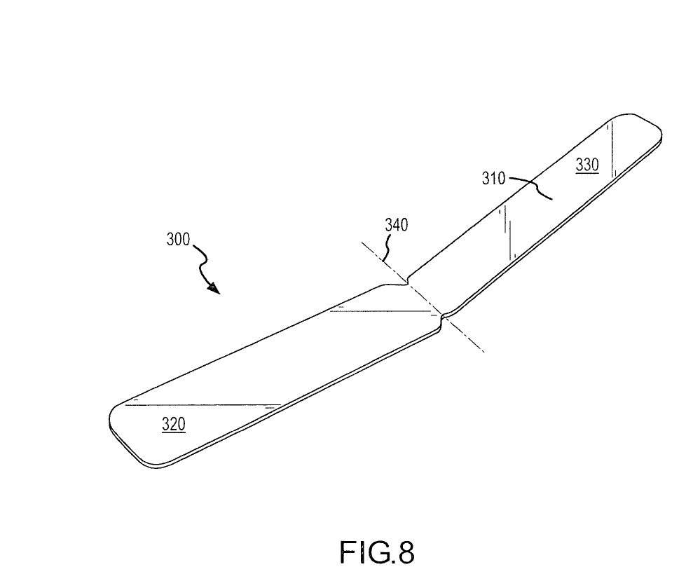

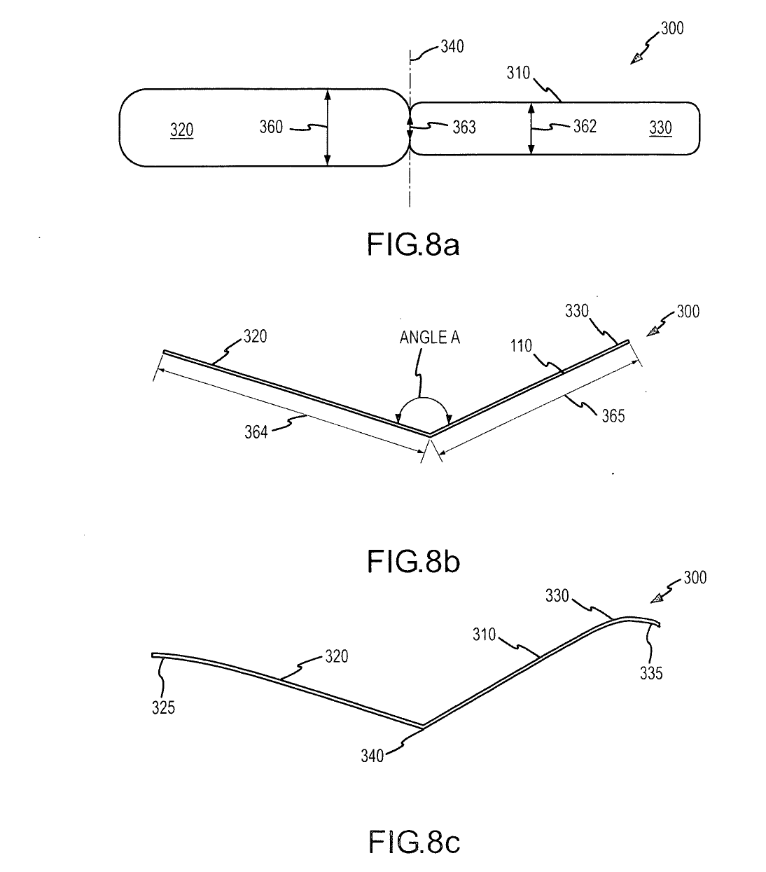

FIG. 8 is a perspective view of an exemplary lingual mirror 300. FIG. 8a is a top plan view of the lingual mirror 300 shown in FIG. 8. FIG. 8b is a side elevation view of the exemplary lingual mirror 300 shown in FIG. 8.

As with the buccal mirror 200, it is again noted that many features described above for the occlusal mirror 100 are also applicable to the lingual mirror 300, and therefore the descriptions are not repeated herein. Instead, the following discussion explains features of the lingual mirror 300 while incorporating the above discussion of the occlusal mirror 100 and buccal mirror 200 by reference. It is also noted that 300-series references are used to refer to like elements designated by the 100-series and 200-series references above, and may not be fully described again with regard to the lingual mirror 300.

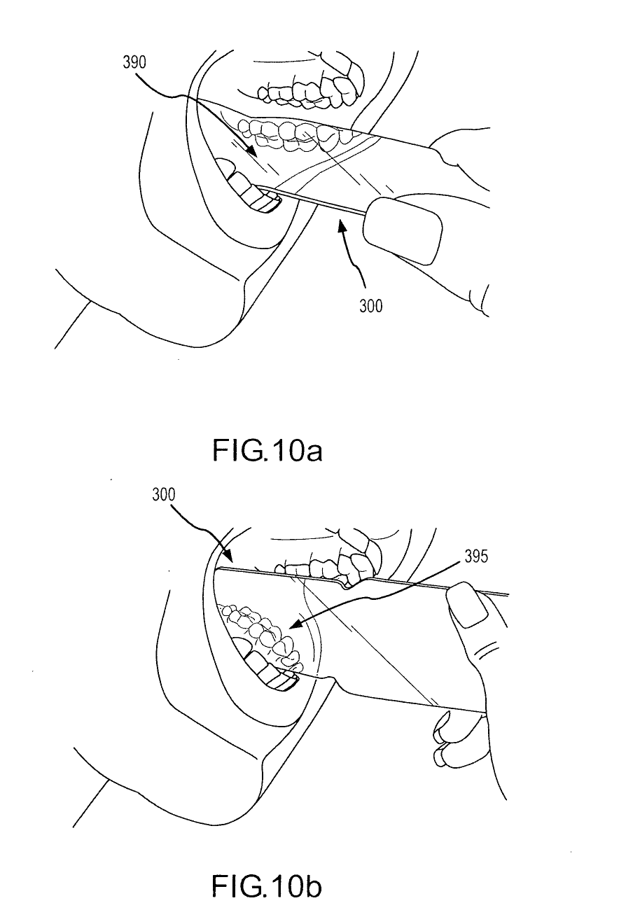

As described above for the occlusal mirror 100, lingual mirror 300 may comprise a body 310 with first and second lobes 320, 330. In an exemplary embodiment, the first and second lobes of the lingual mirror 300 are substantially rectangular shape, (e.g., as can be seen in FIGS. 8 and 8a) to fit inside the patient's mouth (see, e.g., FIGS. 10a and 10b).

For purposes of illustration, an exemplary interchangeable adult male and pediatric occlusal mirror may have the following dimensions. With reference to FIG. 8a, the width of first lobe 320 (referred to by dimension arrows 360) is about one inch. The width of second lobe 330 (referred to by dimension arrows 362) may be about three-quarter inches. The width at the bend indicated by line 340 in FIGS. 8 and 8a (referred to by dimension arrows 363) may be about five-eighths inches. With reference now to FIG. 8b, the length of first lobe 320 (indicated by dimension arrows 364) may be about three and seven-eighths inches and the length of second lobe 330 (indicated by dimension arrows 365) may be about three and seven-eighths inches. Angle A is about 135 degrees.

As with the occlusal mirror 100, it is noted that other embodiments of the lingual mirror 300 are not limited to any particular shape, size, or angle A, and do not need to be interchangeable between different sizes.

Although not shown, in other embodiments the lingual mirror 300 may also include indentations or notches similar to those described above for the occlusal mirror 100. Likewise, the lingual mirror 300 may be symmetrical about a longitudinal centerline, as shown in the figures. In alternative embodiments, however, the lingual mirror 300 may includes features which are offset from the longitudinal centerline similarly to that described above for the occlusal mirror 100, e.g., to enhance use as a retractor and provide a better grip for right-handed or left-handed users.

In addition, the first and second lobes 320, 330 of lingual mirror 300 may be substantially planar, as can be seen in FIG. 8b, or curved at the outermost edges indicated at 325 and 335, respectively, as shown in an alternative embodiment of the buccal mirror in FIG. 8c.

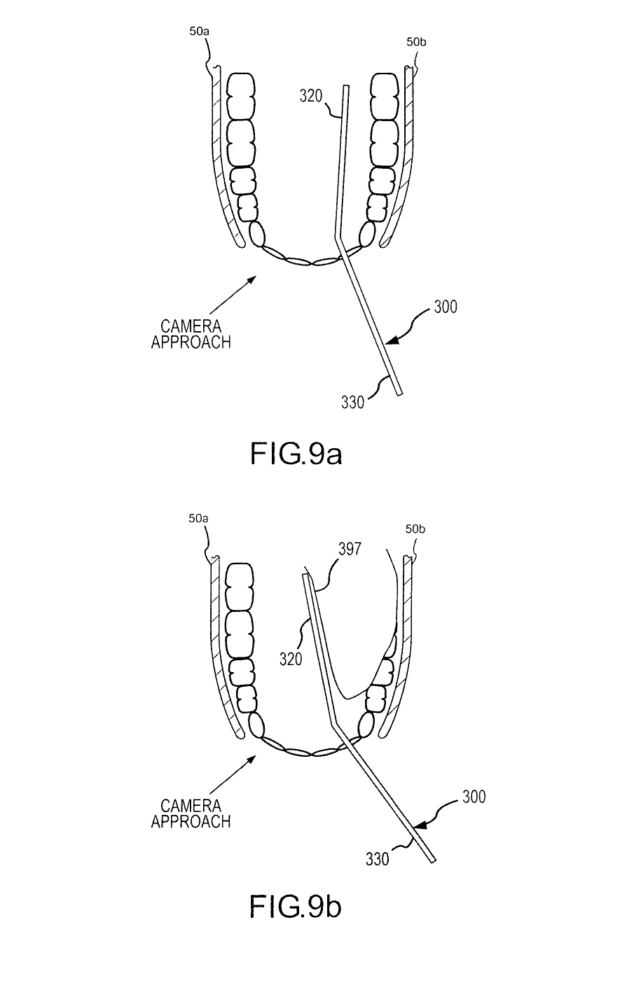

FIGS. 9a and 9b are diagrammatic plan views of a patient's mouth illustrating implementation of an exemplary lingual mirror as it may be used for photographing a patient's teeth in (a) the top-right quadrant, and (b) the bottom-right quadrant. FIGS. 10a and 10b are perspective views of a person's mouth, illustrating an exemplary lingual mirror as it may be used for photographing the inner side-surface of a patient's teeth in (a) the top-right quadrant, and (b) the bottom-right quadrant.

Lingual views are views of the left-inside and right-inside surfaces of a patient's teeth (and surrounding gum and other parts of the mouth), e.g., as indicated in FIG. 1 by arrows 14a and 24a (right-inside) and arrows 14b and 24b (left-inside).

In use, the user (e.g., dentist, dental technician or even the patient) may hold the lingual mirror 300 vertically by one of the lobes 320 or 330, and insert the other lobe (i.e., the imaging surface) into the patient's mouth between the tongue 397 (FIG. 9b) and the patient's teeth. The lobe that is inserted into the patient's mouth may also be used to retract the patient's tongue 397 (and optionally cheeks 50a, 50b) so that it does not interfere with the reflected image. The imaging surface reflects a lingual view of the patient's teeth (and surrounding gums and mouth). The bend automatically positions the user's fingers such that they do not interfere with the image being reflected by the imaging surface. The user does not have to maneuver his or her hand out of the way of the camera approach.

The user (or another user such as an assistant) may position a camera adjacent the lingual mirror 300 to photograph the image being reflected by the lingual mirror 300, as illustrated by the camera approach shown in FIGS. 9a and 9b. The lingual mirror 300 may be used to obtain separate lingual views of each quadrant (e.g., quadrants 10a, 10b, 20a, and 20b in FIG. 1) by having the patient open his or her mouth and holding the lingual mirror 300 opposite each quadrant for separate images.

It is noted that directional references and designations such as “first” and “second” as used herein are used for the convenience of the reader and not intended to limit the scope of the invention in any way. It is also noted that while the mirrors were discussed as they may be used by a dentist to photographs a patient's teeth, the mirrors may also have other applications.

In addition to the specific implementations explicitly set forth herein, other aspects and implementations will be apparent to those skilled in the art from consideration of the specification disclosed herein. It is intended that the specification and illustrated implementations be considered as examples only.

1. An intra-oral mirror, comprising:
a first lobe providing an imaging surface during use, the first lobe having a reflective surface for reflecting a photo-quality image of the patient's teeth in the direction of a camera approach during use; and
a second lobe providing a handle, the first and second lobes connected to one another at an angle that automatically positions a user's hand on the handle away from the camera approach during use.

2. The intra-oral mirror of claim 1, wherein the first and second lobes are substantially the same shape.

3. The intra-oral mirror of claim 1, wherein the first and second lobes are different sizes to fit in a plurality of different size and shape mouths.

4. The intra-oral mirror of claim 1, wherein the first lobe is interchangeable as the handle and the second lobe is interchangeable as the imaging surface.

5. The intra-oral mirror of claim 1, wherein the first and second lobes are operable as a retractor to open the patient's mouth and move the patient's tongue before photographing the patient's teeth.

6. The intra-oral mirror of claim 1, wherein both a top surface and a bottom surface of the first and second lobes are reflective.

7. The intra-oral mirror of claim 1, wherein the first and second lobes are connected to one another to form a substantially figure-eight shape for occlusal imaging and lingual imaging.

8. The intra-oral mirror of claim 1, wherein the first and second lobes are a substantially rectangular shape for lingual imaging.

9. The intra-oral mirror of claim 1, wherein the first and second lobes are a substantially oval shape for buccal imaging.

10. The intra-oral mirror of claim 1, wherein the first and second lobes are indented, the indent on the imaging surface allowing the patient's mouth to partially close for the patient's comfort without blocking the photo-quality image of the patient's teeth, and the indent on the handle enhancing the user's grasp of the handle.

11. The intra-oral mirror of claim 1, wherein symmetry of the first and second lobes is offset relative to a longitudinal centerline through the first and second lobes.

12. The intra-oral mirror of claim 11, wherein the offset in symmetry facilitates either right-handed or left-handed users to insert the imaging surface at least partially in the patient's mouth.

13. A method, comprising:
providing a mirror for partial insertion into a patient's mouth, the mirror having opposing lobes oriented at an angle relative to one another such that a user's hand is positioned away from a camera approach during use even if the user is holding the mirror in the patient's mouth; and
reflecting an image of a patient's teeth from one of the opposing lobes toward the camera approach when the other opposing lobe is positioned in the patient's mouth adjacent the patient's teeth, the image of the patient's teeth suitable for photographing.

14. The method of claim 13, further comprising selecting different sizes for the opposing lobes so that the same mirror can be used in different size mouths.

15. The method of claim 13, further comprising shaping at least one of the opposing lobes for either right-handed users or left-handed users.

16. The method of claim 13, further comprising forming a notch in at least one of the opposing lobes to facilitate entry and exit of the notched lobe into and out of the patient's mouth.

17. The method of claim 13, further comprising forming a notch in at least one of the opposing lobes to enhance the user's grasp of the notched lobe.

18. A mirror for photographing teeth, comprising:
first lobe means for positioning and holding second lobe means adjacent the teeth, a plane through the first lobe means angled relative to a plane through the second lobe means so that a user holding the first lobe means does not block a camera approach adjacent the second lobe means during use; and
imaging means for reflecting a photo-quality image of the teeth from the second lobe means in a direction of the camera approach during use.

19. The mirror of claim 18, wherein the first and second lobe means are the same shape as one another and different sizes from one another so that the same mirror can be used with different sizes and shapes of mouths.

20. The mirror of claim 18, wherein the first and second lobe means are interchangeable for use.
